(19) United States
(12) Patent Application Publication
(10) Pub. No.: US 2024/0096452 A1
(43) Pub. Date: Mar. 21, 2024

(54) MOLECULAR STRUCTURE OPTIMIZATION SYSTEM, MOLECULAR STRUCTURE OPTIMIZATION METHOD, AND PARAMETERIZED QUANTUM CIRCUIT

(71) Applicant: KABUSHIKI KAISHA TOSHIBA, Tokyo (JP)

(72) Inventors: Yasutaka NISHIDA, Tama Tokyo (JP); Fumihiko AIGA, Kawasaki Kanagawa (JP)

(73) Assignee: KABUSHIKI KAISHA TOSHIBA, Tokyo (JP)

(21) Appl. No.: 18/174,683

(22) Filed: Feb. 27, 2023

(30) Foreign Application Priority Data
Sep. 20, 2022 (JP) 2022-149218

(51) Int. Cl. G16C 10/00; G06N 10/20

(52) U.S. Cl. CPC G16C 10/00; G06N 10/20

(57) ABSTRACT

A molecular structure optimization system includes a quantum computer and a classical computer. The quantum computer uses a parameterized quantum circuit to calculate a loss function from a coordinate parameter of a target molecule. The classical computer updates the coordinate parameter and the circuit parameter based on the loss function, and determines optimum values of the circuit parameter and the coordinate parameter. The classical computer updates a provisional value of the circuit parameter while fixing the coordinate parameter and changing the circuit parameter. The classical computer updates a provisional value of the coordinate parameter while fixing the circuit parameter and changing the coordinate parameter.

### CROSS-REFERENCE TO RELATED APPLICATION

This application is based upon and claims the benefit of priority from Japanese Patent Application No. 2022-149218, filed Sep. 20, 2022, the entire contents of which are incorporated herein by reference.

### FIELD

Embodiments described herein relate generally to a molecular structure optimization system, a molecular structure optimization method, and a parameterized quantum circuit.

### BACKGROUND

In recent years, the development of gate-type quantum computers has progressed significantly, and quantum computing using quantum properties can be implemented by various methods although it is on a small scale. These quantum computers are called noisy intermediate-scale quantum (NISQ) devices, and are regarded as an important first step of a milestone for a quantum computer implementing future error correction. Research utilizing NISQ is currently actively conducted, and in particular, an algorithm called variational quantum eigensolver (VQE) (see Non-Patent Literature 1 (A. Peruzzo, J. McClean, P. Shadbolt, M.-H. Yung, X.-Q. Zhou, P. J. Love, A. Aspuru-Guzik, and J. L. O'Brien, “A variational eigenvalue solver on a photonic quantum processor,” Nature Communications, 5, article number: 4213, 2014.)) is expected to be applied to quantum chemistry computing as a method of hybridizing and utilizing a quantum computer and a classical computer. However, in order to actually execute the algorithm on an NISQ device, it is necessary to prepare a parameterized quantum circuit (PQC) called an ansatz corresponding to a variational trial function, calculate an expected value of a Hamiltonian inserted in the ansatz on the NISQ device, and update the circuit parameter of the ansatz using a classical computer so that the expected value becomes small. That is, in the VQE, it is necessary to repeatedly calculate the expected value and update the parameter until the expected value to be obtained becomes sufficiently small while alternately performing processing in the NISQ device and the classical computer many times. In order to perform the VQE calculation with high accuracy, it is necessary to improve various problems with a trial function with high expressivity, an ansatz, an optimization method (optimizer) with high performance, an efficient sampling method, mitigation (error mitigation) of a noise error inherent in the NISQ device, circuit design based on actual architecture (qubit layout), and the like.

### BRIEF DESCRIPTION OF THE DRAWINGS

FIG. 1 is a block diagram illustrating an example of a configuration of a molecular structure optimization system.

FIG. 2 is a block diagram related to molecular structure optimization processing.

FIG. 3 is a schematic diagram of the molecular structure optimization processing.

FIG. 4 is a diagram illustrating a processing procedure of the molecular structure optimization processing.

FIG. 5 is a schematic diagram of molecular structure optimization processing according to a comparative example.

FIG. 6 is a graph illustrating total energy according to the comparative example.

FIG. 7 is a graph illustrating an energy difference from energy in a most stable structure according to the comparative example.

FIG. 8 is a graph illustrating total energy according to an example.

FIG. 9 is a graph illustrating an energy difference from energy in a most stable structure according to the example.

FIG. 10 is another graph illustrating total energy according to the example.

FIG. 11 is another graph illustrating an energy difference from energy in the most stable structure according to the example.

### DETAILED DESCRIPTION

Since the VQE can be used for a minimum eigenvalue problem, application to quantum chemistry computing for calculating a ground state of a molecular system is most expected. In recent years, Non-Patent Literature 2 (A. Delgado, J. M Arrazola, S. Jahangiri, Z. Niu, J. Izaac, C. Roberts, and N. Killoran, “Variational quantum algorithm for molecular geometry optimization” Phys. Rev. A 104, 052402 (2021)) has disclosed a report that a molecular structure can be optimized by calculating not only ground-state energy but also a force acting on an atom. However, in order to obtain the ground-state energy by the VQE, it is necessary to repeatedly perform calculation to optimize a parameter of a quantum circuit by alternately performing processing in a classical computer and a quantum computer many times. For this reason, in a case where the structure optimization is performed using a Helman-Feynman force calculated from the gradient of the ground-state energy with respect to atomic coordinates, an enormous number of iterative calculations including calculation processes for energy minimization and structure optimization are required. Therefore, in order to perform practical calculation for optimizing a molecular structure using a quantum computer, it is essential to reduce the number of iterative calculations of the VQE.

Regarding a series of studies on reducing the calculation cost of the VQE, lannelli & Jansen (Non-Patent Literature 3 (G. lannelli and K. Jansen, “Noisy Bayesian optimization for variational quantum eigensolvers,” arXiv 2112.00426)) in 2021 and Rad et al. (Non-Patent Literature 4 (A. Rad, A. Seif, and N. Linke, “Surviving The Barren Plateau in Variational Quantum Circuits with Bayesian Learning Initialization,” arXiv 2203.02464, vl.)) in 2022 have disclosed a method of using Bayesian optimization as an optimization method. According to Non-Patent Literatures 3 and 4, it has been reported that when Bayesian optimization is used, an optimum value of total energy can be obtained with fewer parameter updates than conventional optimization methods, and it has been reported that the barren plateaus problem in which convergence worsens with the circuit depth of a parameterized quantum circuit can be alleviated by variational optimization in a NISQ device. As described above, it has been reported that Bayesian optimization is effective for variational optimization for energy based on the VQE, but it is unclear whether Bayesian optimization is similarly effective for optimization of a molecular structure.

An efficient method for molecular structure optimization using the VQE is disclosed in Non-Patent Literature 2. According to Non-Patent Literature 2, it has been reported that the total energy is optimized in the vector space spanned by the circuit parameters and the atomic coordinates, and high efficiency can be achieved by sequentially optimizing the circuit parameters and the atomic coordinates without directly computing the Helman-Feynman force. However, the optimization method disclosed in Non-Patent Literature 2 is based on a conventional gradient method, and cannot be said to be very efficient.

A molecular structure optimization system according to an embodiment includes a quantum computing unit, an update unit, and an optimization unit. The quantum computing unit calculates a loss function from a coordinate parameter of a processing target molecule by using a parameterized quantum circuit defined by a circuit parameter. The update unit updates the coordinate parameter and the circuit parameter based on the loss function. The optimization unit repeats a variational optimization procedure including the calculation of the loss function by the quantum computing unit and the update of the coordinate parameter and the circuit parameter by the update unit until a stop condition is satisfied, and determines an optimum value of the circuit parameter and an optimum value of the coordinate parameter that minimize or maximize the loss function. The update unit includes a first update unit and a second update unit. The first update unit estimates a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function. The second update unit updates the first provisional value of the first parameter while fixing the second parameter to the second provisional value and changing the first parameter according to the Bayesian optimization algorithm based on the loss function.

Hereinafter, the molecular structure optimization system, a molecular structure optimization method, a molecular structure optimization program, and the parameterized quantum circuit according to the present embodiment will be described with reference to the drawings.

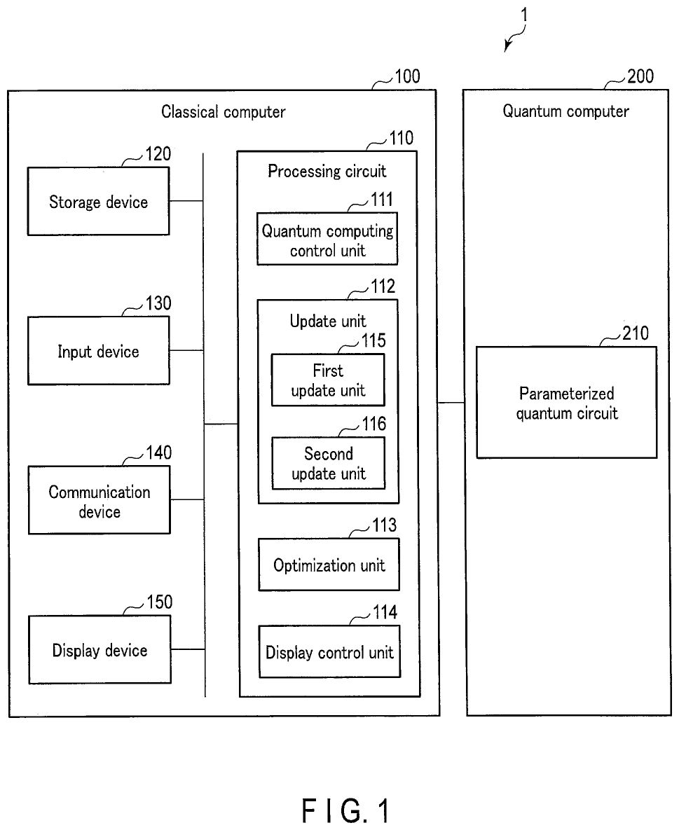

FIG. 1 is a block diagram illustrating an example of a configuration of a molecular structure optimization system 1 according to the present embodiment. As illustrated in FIG. 1, the molecular structure optimization system 1 includes a classical computer 100 and a quantum computer 200. The classical computer 100 and the quantum computer 200 are connected to each other so as to be able to communicate information with each other by wire or wirelessly.

The classical computer 100 is a computer that processes binary classical bits. The classical computer 100 is a computer including a processing circuit 110, a storage device 120, an input device 130, a communication device 140, and a display device 150. Information communication is performed between the processing circuit 110, the storage device 120, the input device 130, the communication device 140, and the display device 150 via a bus. Therefore, since each of the devices is connected to the other devices via information communication, the plurality of devices may be collectively referred to as the classical computer 100. Note that the storage device 120, the input device 130, the communication device 140, and the display device 150 are not essential components, and can be omitted as appropriate.

The processing circuit 110 includes a processor such as a central processing unit (CPU) and a memory such as a random access memory (RAM). The processing circuit 110 includes a quantum computing control unit 111, an update unit 112, an optimization unit 113, and a display control unit 114. The processing circuit 110 implements each function of each of the units 111 to 114 by executing a molecular structure optimization program. The molecular structure optimization program is stored in a non-transitory computer-readable recording medium such as the storage device 120. The molecular structure optimization program may be implemented as a single program that describes all the functions of the units 111 to 114 described above, or may be implemented as a plurality of modules divided into several functional units. Each of the units 111 to 114 may be implemented by an integrated circuit such as an application specific integrated circuit (ASIC)) or a field programmable gate array (FPGA). In this case, the units 111 to 114 may be mounted on a single integrated circuit, or may be individually mounted on a plurality of integrated circuits.

The quantum computing control unit 111 controls quantum computing using a parameterized quantum circuit 210 implemented in the quantum computer 200. The quantum computing control unit 111 calculates a loss function from a coordinate parameter of a processing target molecule using the parameterized quantum circuit 210 defined by a circuit parameter. Specifically, the quantum computing control unit 111 provides the coordinate parameter to the parameterized quantum circuit 210. The coordinate parameter is converted into the loss function by the parameterized quantum circuit 210. The quantum computing control unit 111 acquires the loss function from the parameterized quantum circuit 210.

The update unit 112 updates the coordinate parameter and the circuit parameter based on the loss function. The update unit 112 includes a first update unit 115 and a second update unit 116. The first update unit 115 estimates a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function. The second update unit 116 updates the first provisional value of the first parameter while fixing the second parameter to the second provisional value and changing the first parameter according to the Bayesian optimization algorithm based on the loss function.

The optimization unit 113 repeats a variational optimization procedure including the calculation of the loss function by the quantum computing control unit 111 and the update of the coordinate parameter and the circuit parameter by the update unit 112 until a stop condition is satisfied, and determines an optimum value of the circuit parameter and an optimum value of the coordinate parameter that minimize or maximize the loss function. The optimization unit 113 can determine the optimum value of the coordinate parameter when the processing target molecule has a ground-state molecular structure by minimizing the loss function. The optimization unit 113 can determine the optimum value of the coordinate parameter when the processing target molecule has a transition-state molecular structure by maximizing the loss function.

The display control unit 114 causes the display device 150 to display various types of information. For example, the display control unit 114 causes the display device 150 to display the coordinate parameter, the circuit parameter, the loss function, and the like.

The storage device 120 includes a read only memory (ROM), a hard disk drive (HDD), a solid state drive (SSD), an integrated circuit storage device, or the like. The storage device 120 stores the molecular structure optimization program and the like.

The input device 130 inputs various commands from an operator. As the input device 130, a keyboard, a mouse, various switches, a touch pad, a touch panel display, and the like can be used. An output signal from the input device 130 is supplied to the processing circuit 110. Various commands from the operator may be input not by the input device 130 included in the classical computer 100 but by an input device provided in another classical computer connected via the communication device 140.

The communication device 140 is an interface for performing information communication with an external device such as the quantum computer 200 connected to the classical computer 100 by wire or wirelessly.

The display device 150 displays various types of information under control by the display control unit 114. As the display device 150, a cathode-ray tube (CRT) display, a liquid crystal display, an organic electroluminescence (EL) display, a light-emitting diode (LED) display, a plasma display, or any other display known in the art can be appropriately used. Furthermore, the display device 150 may be a projector.

The quantum computer 200 is a computer that has the quantum circuit 210 implemented therein that performs a quantum gate operation on a plurality of qubits, and performs quantum computing using the quantum circuit 210. As a method of implementing a qubit and a quantum gate by the quantum circuit 210, any method such as a superconducting circuit method, an ion trap method, a quantum dot method, or an optical lattice method may be used. It is assumed that the quantum computer 200 has various types of hardware for implementing an environment according to the method of implementing a qubit and a quantum gate. Although not illustrated in FIG. 1, the quantum computer 200 may include a storage device, an input device, a communication device, a display device, and the like in addition to a processing circuit for performing various types of information processing using classical bits. The quantum computer 200 is an example of the quantum computing unit.

The quantum computer 200 receives the coordinate parameter of the processing target molecule from the classical computer 100, inputs the coordinate parameter to the parameterized quantum circuit 210 that performs a quantum gate operation on a plurality of qubits, and outputs the loss function defined by the coordinate parameter and the circuit parameter. The quantum computer 200 acquires the loss function from the quantum circuit 210. The quantum computer 200 transmits the acquired loss function to the classical computer 100. The quantum computer 200 is an example of the quantum computing unit.

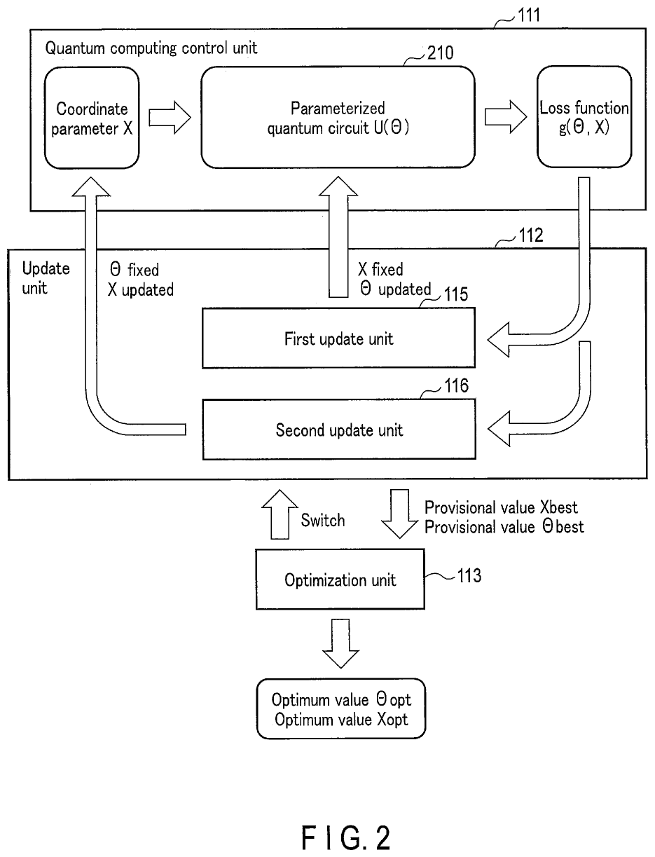

FIG. 2 is a block diagram related to molecular structure optimization processing according to the present embodiment. As illustrated in FIG. 2, the quantum computing control unit 111 inputs a coordinate parameter X to the parameterized quantum circuit 210 controlled by a circuit parameter Θ, and outputs a loss function g(Θ, X). The coordinate parameter X is a vector of coordinates of an atom included in the processing target molecule. The parameterized quantum circuit 210 includes a sequence of quantum gates controlled by the circuit parameter Θ. The quantum gates are assigned the circuit parameter Θ and perform a quantum gate operation according to the circuit parameter Θ on a qubit. The circuit parameter Θ is a rotation angle vector of the quantum gates constituting the parameterized quantum circuit 210. The loss function g(Θ, X) is a Hamiltonian defined by the coordinate parameter X of the processing target molecule. The parameterized quantum circuit 210 is controlled by the circuit parameter Θ, and thus can be expressed by a mathematical expression such as U(Θ).

The parameterized quantum circuit 210 performs the quantum gate operation according to the circuit parameter Θ on a qubit encoded with the coordinate parameter X, and outputs the loss function g(Θ, X) representing energy in a desired quantum state.

The first update unit 115 estimates a provisional value of the circuit parameter Θ while fixing the coordinate parameter X, which is the first parameter, to the provisional value and changing the circuit parameter Θ, which is the second parameter, according to the Bayesian optimization algorithm based on the loss function g(Θ, X). Specifically, the first update unit 115 sets the circuit parameter Θ to a candidate point Θs1 while fixing the coordinate parameter X to a provisional value Xbest, and calculates a loss function g(Θs1, Xbest) using the parameterized quantum circuit 210. Next, the first update unit 115 estimates the next candidate point Θs2 based on the loss function g(Θs1, Xbest) according to the Bayesian optimization algorithm. Then, the first update unit 115 sets the circuit parameter Θ to the candidate point Θs2 while fixing the coordinate parameter X to the provisional value Xbest, and calculates a loss function g(Θs2, Xbest) using the parameterized quantum circuit 210. Next, the first update unit 115 estimates the next candidate point Θs3 based on the loss function g(Θs2, Xbest) according to the Bayesian optimization algorithm. In this way, the estimation of a candidate point Θsl (l is a natural number of L or less) and the calculation of a loss function g(Θsl, Xbest) are repeated L times. This iterative processing is referred to as Bayesian updating. After the iterative processing is performed L times, a provisional value Θbest of the circuit parameter Θ that minimizes or maximizes the loss function g in the iterative processing performed L times is determined.

The second update unit 116 updates the provisional value of the coordinate parameter X while fixing the circuit parameter Θ to the provisional best and changing the coordinate parameter X according to the Bayesian optimization algorithm based on the loss function g(Θ, X). Also in the second update unit 116, the estimation of a candidate point Xsm (m is a natural number of M or less) and the calculation of a loss function g(Θbest, Xsm) are repeated M times. After the iterative processing is performed M times, a provisional value Xbest of the coordinate parameter X that minimizes or maximizes the loss function g in the iterative processing performed M times is determined.

The optimization unit 113 repeats a variational optimization procedure including the calculation of the loss function g(Θ, X) by the quantum computing control unit 111 and the update of the coordinate parameter X and the circuit parameter Θ by the update unit 112 until a stop condition is satisfied, and determines an optimum value Θopt of the circuit parameter Θ and an optimum value Xopt of the coordinate parameter X that minimize or maximize the loss function. As the variational optimization procedure, for example, a variational quantum eigenvalue method (VQE) is used.

The molecular structure optimization processing according to the present embodiment will be described below in detail. In the following description, the molecular structure optimization processing according to the present embodiment solves a problem of minimizing a loss function.

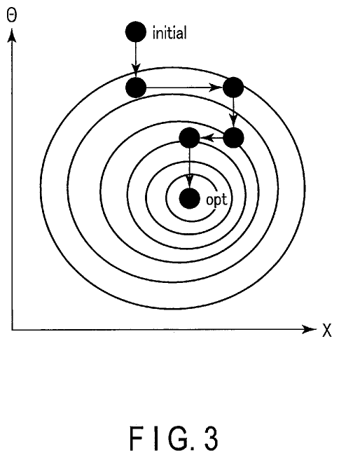

FIG. 3 is a schematic diagram of the molecular structure optimization processing according to the present embodiment. As illustrated in FIG. 3, the molecular structure optimization method according to the present embodiment is synonymous with searching for the minimum value of an energy curved surface E(Θ, X) defined in a vector space including the circuit parameter Θ=(θ₀, θ₁, θ₂, . . . , θₙ) of the parameterized quantum circuit 210 and the coordinate parameter X=(x₀, x₁, x₂, . . . , xₙ) of the molecular structure. FIG. 3 illustrates the energy curved surface E(Θ, X) expressed by energy contour lines. Each black spot is a vector (Θk, Xₖ) in each optimization step, and is expressed as one point on the energy curved surface E(Θ, X). The arrows represent the progression of the optimization steps. FIG. 3 illustrates a state in which the optimization steps are sequentially performed in the Bayesian optimization in the order of Θ, X, and Θ from the point of initial which is the initial vector when the optimization is started, and finally the minimum value opt of the energy curved surface E(Θ, X) is reached.

Next, the variational quantum eigenvalue method (VQE) will be described. The VQE performs calculation according to a variational optimization algorithm using a quantum circuit according to the following procedures.
Procedure 1. Embedding of a wave function (trial function) in a qubit
Procedure 2. Transformation of a Hamiltonian quantized by the second quantization method into a qubit Hamiltonian
Procedure 3. Calculation of an expected value using an NISQ device
Procedure 4. Update of a parameter using a classical computer
Procedure 5. Repeat procedures 3 and 4 until convergence

In the VQE, since the trial function is calculated on the quantum circuit, it is necessary to embed the trial function in the qubit (procedure 1). Specifically, when the state “0” of the qubit is regarded as an unoccupied orbit and the state “1” of the qubit is regarded as an occupied orbit, the second quantization method expressing the quantum state in the occupied state of the spin orbit can be applied as it is. However, it is impossible to directly calculate the Hamiltonian quantized by the second quantization method on the quantum circuit, and the Hamiltonian quantized by the second quantization method is rewritten by a gate that performs a qubit operation, that is, by using a Pauli operator (procedure 2). As a representative method of expressing the Hamiltonian by a linear combination of the Pauli operator, there is a Jordan-Wigner transformation (Jordan, P. and Wigner, E. 1928 “Uber das Paulische Aquivalenzverbot,” Zeitschrift fur Physik 631), and as some other transformation methods, a Bravi-Kitaev transformation (Bravi, S. and Kitaev, A. 2005 “Universal Quantum Computation with ideal Cliffordgates and noisy ancillas,” Phys. Rev. A 022316) and the like are known. By using these transformation methods, the Hamiltonian is expressed by the following Equation (1).

$$ℋ = {\sum }_{k} {h}_{k} {P}_{k} = {\sum }_{k} {h}_{k} {\prod }_{k} {σ}_{l}^{k} \qquad ( 1 )$$

In Equation (1), σₗϵ{I, X, Y, Z}, which are an identity operator and Pauli operators of X, Y, and Z components, respectively. For example, as expressed in Equation (2), when a parameterized quantum circuit U(Θ) including a unitary operator is caused to act on the initial quantum state, the quantum state ψ(Θ) is obtained.

$${{ψ ( Θ ) = U ( Θ ) \vert 0 〉}^{{⊗}_{n}} \\ \vert 0 〉}^{{⊗}_{n}} : Initial quantum state \qquad ( 2 )$$

When the Hamiltonian described in the above Equation (1) is inserted with the quantum state ψ(Θ) as a bra-ket vector, the expected value can be calculated (procedure 3). The variational optimization regarding the expected value is performed via the circuit parameter. The variational optimization is performed by the classical computer (procedure 4). As described above, in the VQE, while processing is alternately performed in the NISQ device and the classical computer many times, the iterative calculation is performed until the expected value becomes sufficiently small (procedure 5) to obtain ground-state energy.

Next, molecular structure optimization will be described. When the VQE is applied to quantum chemistry computing, the ground-state energy of the molecular system can be calculated. As a result, important reactions for material development, compound stability, and the like can be discussed. In addition, as reported by Sokolov (I. Sokolov, P. Barkoutsos, L. Moeller, P. Suchsland, G. Mazzola, and I. Tavernelli, “Microcanonical and finite-temperature ab initio molecular dynamics simulations on quantum computers,” Physical Review Research 3, No. 1, 013125 (2021).) et al., it is possible to calculate a force F^{HF} acting on an atom as in the following Equation (3) using the ground state obtained by the VQE and using the Helman-Feynman theorem.

$${F}^{HF} ( r ) = - 〈 {Ψ}_{0} \vert \frac{\partial ℋ}{\partial r} \vert {Ψ}_{0} 〉 \qquad ( 3 )$$

In Equation (3), ψ₀ is a ground state in which a parameter is optimized by the VQE. A stable molecular structure can be obtained by minutely displacing an atom until the Helman-Feynman force F^{HF} becomes very small based on the following Equations (4) and (5) using the Helman-Feynman force F^{HF}.

$$r ( t + Δ t ) = {r}_{i} ( t ) + Δ {tv}_{i} ( t + Δ t / 2 ) \qquad ( 4 )$$

$${v}_{i} ( t + Δ t / 2 ) = {v}_{i} ( t - Δ t / 2 ) + \frac{Δ t}{m} {F}^{HF} ( r ) \qquad ( 5 )$$

By using Equations (4) and (5) in this manner, it is possible to stably perform the VQE calculation with high accuracy. This can reduce the number of iterative calculations.

Next, the Bayesian optimization will be described. The Bayesian optimization (J. Snoek, L. Hugo, and R. Adams, “Practical Bayesian optimization of machine learning algorithms,” Advances in neural information processing systems, No. 25, 2951-2959 (2012)) is a method of searching for an input value that gives a maximum value or a minimum value of an output value (y value) from a predictive distribution based on prior information (priorϵD). When this is skillfully used, it is possible to obtain a calculation condition and a process condition for obtaining the minimum value or the maximum value of the desired y value with as few trials as possible in a simulation with high calculation cost or a complex experimental process. In the Bayesian optimization, a surrogate model represented by a Gaussian process regression (GP) is used as a predictive distribution, and a search is performed based on a score value of an acquisition function calculated from an expected value and a variance of the predictive distribution. When the surrogate model is a GP, a predictive distribution obtained after training of the GP is a multivariate Gaussian distribution whose variance-covariance matrix is a Gram matrix. When an unknown input value X′ is close to X included in D, the prediction variance of X′ is small, while the prediction variance of X′ away from X behaves to be large. Therefore, in the Bayesian optimization, an efficient search can be performed by carefully considering a relationship in a trade-off between optimum value selection (utilization) and selection (search) of an unevaluated point having a large standard deviation.

The following description is limited to the case of the GP, and the variance-covariance matrix of the predictive distribution is briefly described below. The Gram matrix is given by the following Equation (6) by preparing an appropriate positive definite kernel function Ker and using XϵD (assuming that N pieces of data are present as prior).

[G_{N}]_{i-1 . . . N,j-1 . . . N}=Ker(Xᵢ,Xⱼ) (6)

Here, assuming that an unknown point X′ is newly added, an expected value E[X′] and a variance Σ[X′] of the unknown point X′ are considered. Assuming that noise is not included in the original data (Xᵢ, yᵢ), the expected value E[X′] and the variance Σ[X′] can be analytically calculated according to the following Equations (7) and (8), respectively, from analytical properties of a conditional multidimensional Gaussian distribution.

E[X′]=Ker(X′,Xᵢ)Ker(Xᵢ,Xⱼ)⁻¹yⱼ (7)

E[X′]=Ker(X′,X′)−Ker(X′,Xᵢ)Ker(Xᵢ,Xⱼ)⁻¹Ker(Xⱼ,X′) (8)

Equation (9) is obtained by rewriting Equation (7) by using Equation (6), and Equation (10) is obtained by rewriting Equation (8) by using Equation (6). Therefore, the expected value and variance of X′ can be calculated by an inverse matrix of the N×N Gram matrix formed in the original D.

E[X′]=Ker(X′,Xᵢ)[G_{N}]ᵢⱼ⁻¹yⱼ (9)

Σ[X′]=Ker(X′,X′)−Ker(X′,Xᵢ)[G_{N}]ᵢⱼ⁻¹Ker(Xⱼ,X′) (10)

In a case where noise occurs in data in Equations (9) and (10), expansion can be performed as expressed by the following Equations (11) and (12) using a unit matrix Iᵢⱼ.

E[X′]=Ker(X′,Xᵢ)([G_{N}]ᵢⱼ+σₙ²Iᵢⱼ)⁻¹yⱼ (11)

Σ[X′]=σₙ²+Ker(X′,X′)−Ker(X′,Xᵢ)([G_{N}]ᵢⱼ+σₙ²Iᵢⱼ)⁻¹Ker(Xⱼ,X′) (12)

A case where no noise is present corresponds to σₙ=0, and similar analysis can be performed even in a case where noise is present. As a representative functional form of the kernel function Ker of Equation (6), the following Equation (13) called a radial basis function (RBF) is simply and often used.

$$Ker ( {X}_{i} , {X}_{j} ) = exp ( - \frac{1}{2 β} {‖ {X}_{i} - {X}_{j} ‖}^{2} ) \qquad ( 13 )$$

The Equation (13) includes a hyperparameter of β, which is determined during learning of the GP.

For the unknown input value X′, a candidate point that may cause the y value to be set to the maximum value or the minimum value can be determined by using the expected value and the variance obtained by using Equations (9) and (10) or Equations (11) and (12). This candidate point can be determined based on a score value of an evaluation function called an acquisition function. Specifically, X′ indicating the highest score value is determined as the next candidate point. There are a plurality of definitions of the acquisition function, and here, a representative probability of improvement (PI) and an expected improvement (EI) used in the following analysis will be described. First, regarding PI, when a maximum output value y in D is ybest, a probability that a predicted value of any X′ exceeds ybest is defined as a score value. The acquisition function α_{PI} of PI can be calculated according to Equation (14).

$${α}_{PI} = {Φ}_{normal} ( \frac{{y}_{best} - E [ {X}^{′} ]}{\sqrt{\sum [ {X}^{′} ]}} ) \qquad ( 14 )$$

Here, ψₙₒᵣₘₐₗ(x) is a cumulative distribution function obtained by integration in a range of [x: ∞] (where x>0) of the normal distribution function φₙₒᵣₘₐₗ(x). The next candidate point can be determined as X′ that maximizes α_{PI}.

Next, regarding EI, an expected value of a difference between a predicted value y′ and ybest for any X′ is defined as a score value. Similarly, the acquisition function α_{EI} of EI can be calculated according to the following Formula (15).

$${α}_{EI} = ( E [ {X}^{′} ] - {y}_{best} ) {Φ}_{normal} ( \frac{{y}_{best} - E [ {X}^{′} ]}{\sqrt{\sum [ {X}^{′} ]}} ) + \sqrt{E [ {X}^{′} ]} {ϕ}_{normal} ( \frac{{y}_{best} - E [ {X}^{′} ]}{\sqrt{\sum [ {X}^{′} ]}} ) \qquad ( 15 )$$

Similar to PI, the next candidate point can be determined as X′ that maximizes α_{EI}.

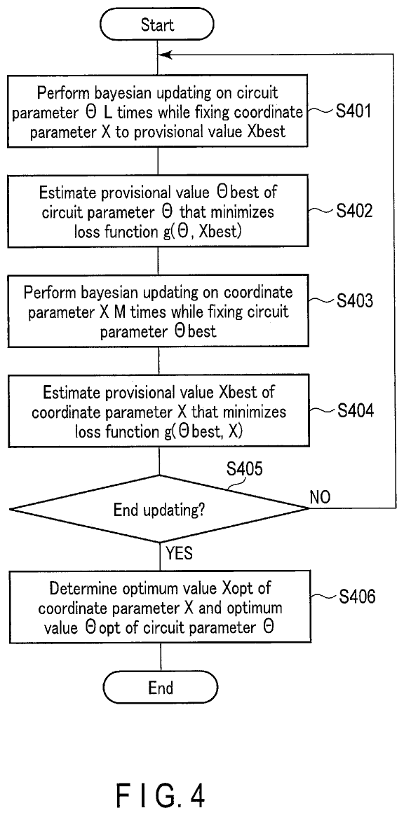

FIG. 4 is a diagram illustrating a processing procedure of the molecular structure optimization processing according to the present embodiment. As illustrated in FIG. 4, the first update unit 115 performs Bayesian updating on the circuit parameter Θ L times while fixing the coordinate parameter X to the provisional value Xbest (step S401). The provisional value (initial value) Xbest in the first update Bayesian updating in step S401 may be set to any value designated by a user or the like. The number L of times of the Bayesian updating may be set to an arbitrary numerical value of 2 or more.

The processing procedure of the Bayesian updating according to step S401 is as follows. First, the quantum computing control unit 111 provides the initial value Xbest of the coordinate parameter X and the candidate point (initial value) Θs1 of the circuit parameter Θ to the parameterized quantum circuit 210 of the quantum computer 200. The initial value Θs1 may be set to an arbitrary value designated by the user or the like, or may be set to a value randomly determined by a random generator or the like. The parameterized quantum circuit 210 performs a quantum gate operation according to the circuit parameter Θs1 on a qubit encoded with the coordinate parameter Xbest, and outputs the loss function g(Θs1, Xbest) representing ground-state energy. The loss function g(Θs1, Xbest) is transferred by the quantum computer 200 to the classical computer 100. The first update unit 115 estimates the next candidate point Θs2 based on the loss function g(Θs1, Xbest) according to the Bayesian optimization algorithm described above. The quantum computing control unit 111 provides the candidate point Θs2 of the circuit parameter Θ to the parameterized quantum circuit 210 of the quantum computer 200. The parameterized quantum circuit 210 performs a quantum gate operation according to the circuit parameter Θs2 on the qubit encoded with the coordinate parameter Xbest, and outputs the loss function g(Θs2, Xbest) representing ground-state energy. The loss function g(Θs2, Xbest) is transferred by the quantum computer 200 to the classical computer 100. The first update unit 115 estimates the next candidate point Θs3 based on the loss function g(Θs2, Xbest) according to the Bayesian optimization algorithm. In this way, the estimation of a candidate point Θs1 (1 is a natural number of L or less) and the calculation of a loss function g(Θs1, Xbest) are repeated L times.

After step S401 is performed, the first update unit 115 estimates a provisional value Θbest of the circuit parameter Θ that minimizes the loss function g(Θ, X) (step S402). Specifically, in step S402, the first update unit 115 identifies a candidate point Θsl that minimizes the loss function g(Θsl, Xbest) among the L candidate points Θsl of the circuit parameter Θ obtained by the iterative processing performed L times. The identified candidate point Θsl is estimated to be the provisional value Θbest. As a result, the Bayesian updating for the circuit parameter Θ ends.

After step S402 is performed, the second update unit 116 performs Bayesian updating on the coordinate parameter X M times while fixing the circuit parameter Θ to the provisional value Θbest estimated in step S402 (step S403). The number M of times of the Bayesian updating may be set to an arbitrary numerical value of 2 or more.

The processing procedure of the Bayesian updating according to step S403 is as follows. First, the quantum computing control unit 111 provides the initial value Xs1 of the coordinate parameter X and the provisional value Θbest of the circuit parameter Θ estimated in step S402 to the parameterized quantum circuit 210 of the quantum computer 200. As the initial value Xs1, the provisional value Xbest used in step S401 may be used. The parameterized quantum circuit 210 performs a quantum gate operation according to the circuit parameter Θbest on a qubit encoded with the coordinate parameter Xs1, and outputs a loss function g(Θbest, Xs1) representing ground-state energy. The loss function g(Θbest, Xs1) is transferred by the quantum computer 200 to the classical computer 100. The second update unit 116 estimates the next candidate point Xs2 based on the loss function g(Θbest, Xs1) according to the Bayesian optimization algorithm described above. The quantum computing control unit 111 provides the candidate point Xs2 of the coordinate parameter X to the parameterized quantum circuit 210 of the quantum computer 200. The parameterized quantum circuit 210 performs a quantum gate operation according to the circuit parameter Θbest on a qubit encoded with the coordinate parameter Xs2, and outputs a loss function g(Θbest, Xs2) representing ground-state energy. The loss function g(Θbest, Xs2) is transferred by the quantum computer 200 to the classical computer 100. The second update unit 116 estimates the next candidate point Xs3 based on the loss function g(Θbest, Xs2) according to the Bayesian optimization algorithm. In this way, the estimation of a candidate point Xsm (m is a natural number of M or less) and the calculation of a loss function g(Θbest, Xsm) are repeated M times.

After step S403 is performed, the second update unit 116 estimates a provisional value Xbest of the coordinate parameter X that minimizes the loss function g(Θbest, X) (step S404). Specifically, in step S404, the second update unit 116 identifies a candidate point Xsm that minimizes the loss function g(Θbest, Xsm) among the M candidate points XΘsm of the coordinate parameter X obtained by the iterative processing performed M times. The identified candidate point Xsm is estimated to be the provisional value Xbest. As a result, the Bayesian updating for the coordinate parameter X ends.

After step S404 is performed, the optimization unit 113 determines whether or not to end the updating (step S405). Specifically, in step S405, the optimization unit 113 determines whether or not the stop condition of the Bayesian updating is satisfied. The stop condition can be set to an arbitrary condition such as a condition that the number of iterations of steps S401 to S404 has reached a predetermined number of times (P times). The predetermined number P of times may be a natural number of 1 or more, and may be set to any value by the user. When the optimization unit 113 determines that the stop condition is not satisfied, that is, when the optimization unit 113 determines that the updating is not to be ended (step S405: NO), steps S401 to S405 are repeated.

The second and subsequent Bayesian updating (steps S401 and S402) for the circuit parameter Θ may be performed in the same manner as described above. However, the provisional value of the coordinate parameter is set to the provisional value Xbest estimated in step S404 in the previous iteration round, and the candidate point Θs1 of the circuit parameter Θ is set to the provisional value Θbest estimated in step S402 in the previous iteration round.

In this manner, steps S401 to S405 are repeated until the optimization unit 113 determines in step S405 that the stop condition is satisfied. Then, when the optimization unit 113 determines in step S405 that the updating is to be ended (step S405: YES), the optimization unit 113 determines the optimum value Xopt of the coordinate parameter X and the optimum value Θopt of the circuit parameter (step S406). In step S406, specifically, the optimization unit 113 determines a provisional value Xbest and a provisional value Θbest that minimize the loss function g from among the P provisional values Xbest and the P provisional values Θbest obtained up to the present time. The determined provisional value Xbest and the determined provisional value Θbest are set to the optimum value Xopt and the optimum value Θopt, respectively. The optimization unit 113 can determine the optimum value of the coordinate parameter when the processing target molecule has the ground-state molecular structure by minimizing the loss function.

Note that the trend of the loss function value with respect to the coordinate parameter X is expected not to change qualitatively although the absolute value changes in a situation where the circuit parameter Θ is not completely optimized. Therefore, in the Bayesian updating for the circuit parameter Θ (steps S401 and S402) and the Bayesian updating for the coordinate parameter X (steps S403 and S404), the necessity of obtaining the provisional values Θbest and Xbest with high accuracy is reduced. Therefore, it is possible to reduce the circuit scale of the quantum circuit 210 without greatly affecting the optimum value Xopt of the coordinate parameter.

As an example, the optimum value Xopt and the optimum value Θopt determined in step S406 may be displayed in an arbitrary layout on the display device 150 by the display control unit 114. As another example, the optimum value Xopt and the optimum value Θopt may be stored in the storage device 120 or may be transferred to another computer via the communication device 140.

The optimum value Θopt may be transferred to the quantum computer 200 and set in the parameterized quantum circuit 210. The parameterized quantum circuit 210 in which the optimum value Θopt is set can receive the input of the coordinate parameter X and output a trial wave function, energy, and the like with relatively high accuracy without performing the Bayesian updating process, the optimization process, and the like again.

Then, the molecular structure optimization processing according to the present embodiment ends.

Some processing can be appropriately added to and removed from the molecular structure optimization processing and some processing of the molecular structure optimization processing can be appropriately changed to an extent not departing from the gist of the invention according to the present embodiment. As an example, it is not essential to display, store, and/or transfer the optimum value Xopt and the optimum value Θopt determined in step S406, and when the optimization unit 113 determines in step S405 that the updating is to be ended (step S405: YES), the molecular structure optimization processing according to the present embodiment may be ended. As another example, the order of the Bayesian updating for the circuit parameter Θ (steps S401 and S402) and the order of the Bayesian updating for the coordinate parameter X (steps S403 and S404) may be reversed. In other words, the first parameter to be updated by the first update unit 115 may be the circuit parameter Θ, and the second parameter to be updated by the second update unit 116 may be the coordinate parameter X. In the above description, the circuit parameter and the coordinate parameter that minimize the loss function are searched for, but a circuit parameter and a coordinate parameter that maximize the loss function may be searched for. This makes it possible to calculate transition-state energy.

In addition, the configuration of the molecular structure optimization system 1 illustrated in FIG. 1 is an example, and the present embodiment is not limited thereto. As an example, the quantum computer 200 and the classical computer 100 may be incorporated in one hardware device. In addition, the storage device, the input device, and the circuit included in the classical computer 100 may not be included in one computer. The storage device, the input device, and the circuit included in the quantum computer 200 may not be included in one computer. In each of the classical computer 100 and the quantum computer 200, the devices may be electrically connected to each other to perform a function as one computer.

### Examples

Next, examples of the molecular structure optimization processing according to the present embodiment will be described below in detail.

A processing target molecule according to an example is an H₂ molecule. Numerical simulation was performed using the electron Hamiltonian of the H₂ molecule. In the example, the Hamiltonian was calculated using existing open source libraries PySCF (The Python-based Simulations of Chemistry Framework, Reference 1 (see Q. Sun, T. C. Berkelbach, N. S. Blunt, G. H. Booth, S. Guo, Z. Li, J. Liu, J. D. McClain, E. R. Sayfutyarova, S. Sharma, S. Wouters, and G. K. Chan, Wiley Interdisciplinary Reviews: Computational Molecular Science 8, e1340 (2017))) and OpenFermion (Reference 2 (see J. R. McClean, K. J. Sung, I. D. Kivlichan, Y. Cao, C. Dai, E. S. Fried, C. Gidney, B. Gimby, P. Gokhale, T. Hner, T. Hardikar, V. Havlek, O. Higgott, C. Huang, J. Izaac, Z. Jiang, X. Liu, S. McArdle, M. Neeley, T. O'Brien, B. O'Gorman, I. Ozdan, M. D. Radin, J. Romero, N. Rubin, N. P. D. Sawaya, K. Setia, S. Sim, D. S. Steiger, M. Steudtner, Q. Sun, W. Sun, D. Wang, F. Zhang, and R. Babbush, (2017), arXiv: 1710.07629)).

The simulation of the quantum circuit was performed using Qiskit (Reference 3 (see G. Aleksandrowicz, T. Alexander, P. Barkoutsos, L. Bello, Y. Ben-Haim, D. Bucher, F. Jose Cabrera-Hernandez, J. Carballo-Franquis, A. Chen, C. Chen, J. Chow, A. Corcoles-Gonzales, A. Cross, A. Cross, A. Cross, J. Cruz-Benito, C. Culver, S. Gonzalez, E. Torre, D, Ding, E. Dumitrescu, I. Duran, P. Eendebak, M. Everitt, I. Sertage, A. Frisch, A. Fuhrer, J. Gambetta, B Gago, J. Gomez-Mosquera, D. Greenberg, I. Hamamura, V. Havlicek, J. Hellmers, L. Herok, H. Horii, S. Hu, T. Imamichi, T. Itoko, A. Javadi-Abhari, N. Kanazawa, A. Karazeev, K. Krsulich, P. Liu, Y. Luh, Y. Maeng, M. Marques, F. Martin-Fernandez, D. McClure, D. McKay, S. Meesala, A. Mezzacapo, N. Moll, D. Rodriguez, G. Nannicini, P. Nation, P. Ollitrault, L. O'Riordan, H. Paik, J. Perez, A. Phan, M. Pistoia, V. Prutyanov, M. Reuter, J. Rice, A. Davila, R. Rudy, M. Ryu, N. Sathaye, C. Schnabel, E. Schoute, K. Setia, Y. Shi, A. Silva, Y. Siraichi, S. Sivarajah, J. Smolin, M. Soeken, H. Takahashi, I. Tavernelli, C. Taylor, P. Taylour, K. Trabing, M. Treinish, W. Turner, D. Vogt-Lee, C. Vuillot, J. Wildstrom, J. Wilson, E. Winston, C. Wood, S. Wood, S. Worner, I. Akhalwaya, C. Zoufalhttps. https://doi.org/10.5281/zenodo.2562111, (2019) An Open-source Framework for Quantum Computing)).

The Bayesian optimization was performed using existing open source libraries GPy (Reference 4 (“GPy: Gaussian process framework in python,” http://github.com/SheffieldML/GPy)) and GpyOpt (“GPyOpt: A Bayesian Optimization framework in python” http://github.com/SheffieldML/GPyOpt).

Hereinafter, for convenience, the method according to the technology of the present example will be referred to as bmgo (Bayesian molecular geometry optimization). Calculation conditions of the structure optimization according to the example were set as follows.
Base function: STO-3G
Spin multiplicity: 2S+1=1, charge=0
Fully active space: CAS(2e, 2o)
Parameterized quantum circuit: hardware-efficient ansatz (depth=1 or 2)

As a benchmark, molecular structure optimization was performed with quantum chemistry computing software PySCF. The total energy was −1.13730605 Hr, atomic positions were “H” (0.0325770423, −0.0000000000, −0.0000000000) and “H” (0.7674229577, −0.0000000000, 0.0000000000), the distance between the hydrogen atoms was 0.734845915 Å, and the energy E=−1.1373 Hr, which were obtained as the most stable structure.

Next, results by bmgo will be described. Calculation conditions of the Bayesian optimization were set as follows.
Surrogate model: GP
Acquisition function: EI
Initial sampling number: 10

First, Bayesian optimization according to a comparative example was applied, and all vectors were optimized using {Θ, X} as an input parameter. Here, regarding the coordinate parameter X, coordinates of one hydrogen atom were fixed to H(0, 0, 0), coordinates of the other were H(x, 0, 0), and in consideration of the symmetry of the molecule, only the x coordinate of one hydrogen atom was used as a variable of the atomic coordinates.

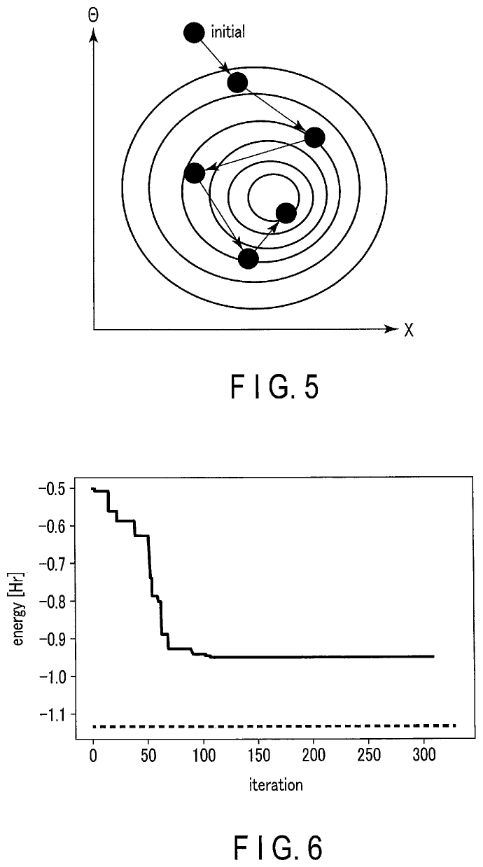

FIG. 5 is a schematic diagram of molecular structure optimization processing according to the comparative example. As illustrated in FIG. 5, unlike the molecular structure optimization processing according to the present embodiment illustrated in FIG. 3, in the molecular structure optimization processing according to the comparative example, both the circuit parameter Θ and the coordinate parameter X are updated in one Bayesian update.

Numerical simulation results of 24 circuit parameters and 1 coordinate parameter (distance between the hydrogen atoms) in a case where D=2 in a hardware-efficient ansatz will be described.

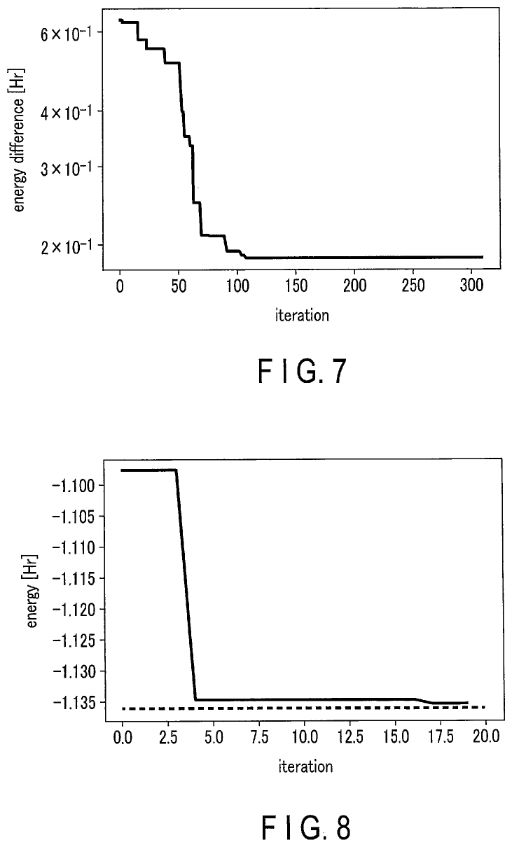

FIG. 6 is a graph illustrating total energy according to the comparative example. FIG. 7 is a graph illustrating an energy difference from energy in a most stable structure according to the comparative example. In FIG. 6, the vertical axis is defined by the total energy [Hr], and the horizontal axis is defined by the number of steps (iterations) of Bayesian updating. In FIG. 7, the vertical axis is defined by a difference (energy difference) [Hr] between the total energy and the energy E=−1.1373 Hr in the most stable structure, and the horizontal axis is defined by the number of steps (iterations) of the Bayesian updating. As illustrated in FIGS. 6 and 7, it can be seen that the energy drop stops in about the 100th step, and the difference from the energy (dotted line) in the most stable structure does not converge well to the optimum solution so far. The final step solution in this case is the total energy E=−0.910093344874445 Hr, the circuit parameter Θ=[3.14159265, −3.14159265, 3.14159265, −3.14159265, −3.14159265, 3.14159265, −3.14159265, 3.14159265, 3.14159265, −3.14159265, −0.11182639, 3.14159265, −3.14159265, 3.14159265, 3.14159265, 3.14159265, −3.14159265, 3.14159265, 3.14159265, −3.14159265, −3.14159265, −3.14159265, −3.14159265, 3.14159265], and the coordinate parameter x=15, and it can be seen that the parameter continues to stay at the search boundary and does not change at all. For confirmation, calculation was performed for several samples, and x=15 was similarly obtained. This is because, in {Θ, X}, the predictive distribution of the expected value of energy cannot be well fitted by the Gaussian process regression, and there is a possibility that the expected value of energy behaves significantly differently for each change in the circuit parameter Θ and the coordinate parameter X. In addition, in general, it is known that multidimensional Bayesian optimization is more likely to cause a local solution, and in a practical molecular system having a larger number of atoms, the Bayesian optimization according to the comparative example illustrated in FIG. 5 is disadvantageous.

Next, results by bmgo that sequentially performs Bayesian optimization as illustrated in FIG. 3 will be described. In bmgo, sequential optimization was performed according to the following procedures.
Procedure 1. Fix the coordinate parameter X and perform Bayesian updating on E(Θ, X) 50 times
Procedure 2. Fix Θ that minimizes E(Θ, X) to the provisional value Θbest and perform Bayesian updating on E(Θbest, X) 50 times
Procedure 3. Fix X that minimizes E(Θbest, X) to the provisional value Xbest
Procedure 4. Repeat procedures 1 to 3 as one step p times
Procedure 5. Determine the optimum value Θopt and the optimum value Xopt

In the present example, an iterative calculation was performed p=20 times in the step of procedure 4. The trend of the energy of the system with respect to the atomic coordinate X is expected not to change qualitatively, although the absolute value changes in a situation where the circuit parameter Θ is not fully optimized. Therefore, it is expected that it is not always necessary to obtain a highly accurate optimum solution at the time of optimization of Θ in procedures 1 and 2, that is, even if the quantum circuit is shallower, the value of Xopt is not greatly affected. Therefore, in order to reduce the calculation load, a hardware-efficient ansatz of D=1 was used in this case.

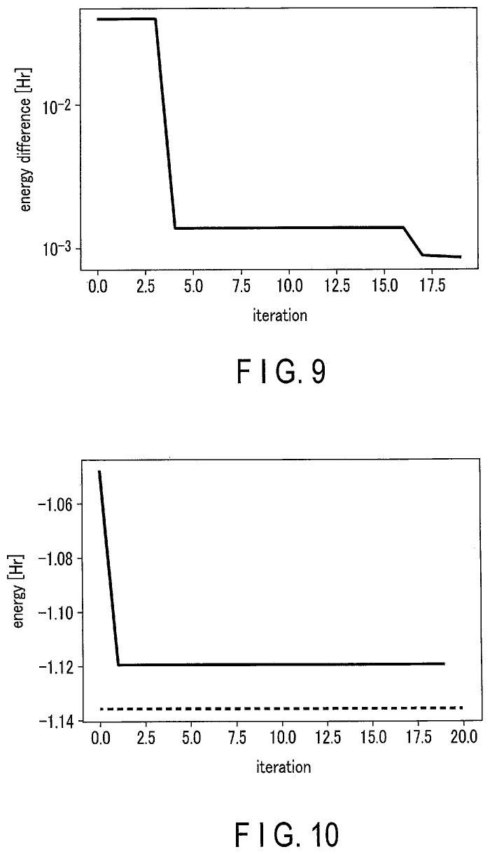

FIG. 8 is a graph illustrating total energy according to the example. FIG. 9 is a graph illustrating an energy difference from energy in the most stable structure according to the example. As illustrated in FIGS. 8 and 9, it can be seen that an energy value close to that in the most stable structure is obtained in about the 6th step in which p>5. In addition, as compared with FIGS. 6 and 7 using the hardware-efficient (HE) ansatz of D=2, it can be seen that a highly accurate expected value of energy is finally obtained. In the final step, the total energy E=−1.135331506112484 Hr, the circuit parameter Θ=[−0.24188404, 3.14159265, −3.14159265, −3.14159265, −1.36057888, −3.14159265, 1.90104979, −3.14159265, −3.14159265, −3.14159265, 3.14159265, −3.14159265, 1.45623973, −3.14159265, 1.49697286, 1.46412561], and the coordinate parameter (distance between the hydrogen atoms) x=0.7344096462252. The distance between the hydrogen atoms is almost the same as the solution (=0.7348) by complete active space configuration interaction (CASCI) of the classical computer with an error of 10⁻⁴, and the energy is also the same with an error on the order of 10⁻³, and it can be seen that the most stable structure is obtained with high accuracy.

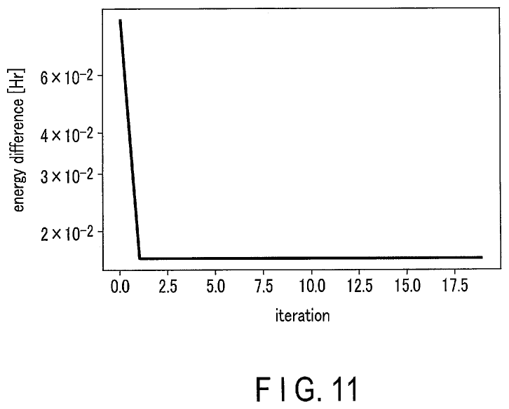

FIG. 10 is another graph illustrating total energy according to the example. FIG. 11 is another graph illustrating an energy difference from energy in the most stable structure according to the example. FIGS. 10 and 11 illustrate results in a case where the energy does not sufficiently converge in 20 steps of the Bayesian optimization. As illustrated in FIGS. 10 and 11, it can be seen that the energy does not completely fall and falls into a local solution. That is, even when the difference from energy in the most stable structure is large, an error of the atomic coordinates of the most stable structure is small. In the final step in this case, the total energy E=−1.119713435544261 Hr, the circuit parameter Θ=[0.27941586, −0.07554233, −0.01952159, −3.14159265, 2.70094774, 0.74961037, −2.08077215, 1.33821656, 3.14159265, 0.28885805, −3.14159265, 3.14159265, −1.54402765, 1.53511816, 3.1024391, −2.6263595], and the coordinate parameter (distance between the hydrogen atoms) x=0.759282304006334. The value of energy is larger than the value (=−1.1373) of the most stable structure, and it can be confirmed that the energy difference is also large. On the other hand, the value of x is 0.7592, which is within an error range of about 3% as compared with a case where the distance between the atoms in the most stable structure is 0.7348. This means that, when the present method is used as described above, even if the circuit parameter Θ is not sufficiently optimized, the optimization itself of the atomic coordinates is not greatly affected. The test calculations using the hydrogen molecule show that sufficiently highly accurate structure optimization is possible with a hardware-efficient ansatz of D=1.

In the above example, optimization of Θ and X was performed so as to minimize E(Θ, X), and a stable molecular structure having the lowest energy was obtained. However, when the present example is used for −E(Θ, X) in which the sign of the cost function is inverted, a molecular structure having the highest energy can be obtained. When the range of the coordinate parameter X is appropriately set, a transition-state molecular structure in a chemical reaction can be obtained. As described above, the present example can be utilized not only in determination of a stable molecular structure but also in search of a transition state in a chemical reaction.

The molecular structure optimization system 1 according to the present embodiment includes the quantum computer 200 and the classical computer 100. The quantum computer 200 calculates a loss function from the coordinate parameter of the processing target molecule using the parameterized quantum circuit 210 defined by the circuit parameter. The classical computer 100 includes the update unit 112 and the optimization unit 113. The update unit 112 updates the coordinate parameter and the circuit parameter based on the loss function. The optimization unit 113 repeats a variational optimization procedure including the calculation of the loss function by the quantum computer 200 and the update of the coordinate parameter and the circuit parameter by the update unit 112 until a stop condition is satisfied, and determines an optimum value of the circuit parameter and an optimum value of the coordinate parameter that minimize or maximize the loss function. The update unit 112 includes the first update unit 115 and the second update unit 116. The first update unit 115 estimates a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function. The second update unit 116 updates the first provisional value of the first parameter while fixing the second parameter to the second provisional value and changing the first parameter according to the Bayesian optimization algorithm based on the loss function.

According to the above configuration, the circuit parameter and the coordinate parameter are optimized using the Bayesian optimization in the molecular structure optimization based on the VQE. Here, instead of performing the Bayesian updating on both the circuit parameter and the coordinate parameter at each time step, the Bayesian updating is alternately performed on the circuit parameter and the coordinate parameter. As a result, a risk of falling into a local solution is reduced as compared with a case where the Bayesian updating is performed on both the circuit parameter and the coordinate parameter, and thus, a reduction in calculation cost and an improvement in accuracy of optimization for the circuit parameter and the coordinate parameter are achieved. In addition, as described above, it is also possible to obtain a highly accurate coordinate parameter and a highly accurate loss function (for example, energy) without sufficiently optimizing the circuit parameter, and thus, it is also possible to reduce the calculation cost of the coordinate parameter and the loss function.

Thus, according to the present embodiment, it is possible to improve the efficiency of calculation in the molecular structure optimization using the parameterized quantum circuit.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Hereinafter, the invention disclosed in the specification and claims at the time of filing of the present application will be appended.

<1>

A molecular structure optimization system includes:
a quantum computer that uses a parameterized quantum circuit defined by a circuit parameter to calculate a loss function from a coordinate parameter of a processing target molecule; and
a classical computer including an update unit that updates the coordinate parameter and the circuit parameter based on the loss function, and an optimization unit that repeats a variational optimization procedure including the calculation of the loss function by the quantum computer and the update of the coordinate parameter and the circuit parameter by the update unit until a stop condition is satisfied, and determines an optimum value of the circuit parameter and an optimum value of the coordinate parameter that minimize or maximize the loss function.

The update unit includes
a first update unit that estimates a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function; and
a second update unit that updates the first provisional value of the first parameter while fixing the second parameter to the second provisional value and changing the first parameter according to the Bayesian optimization algorithm based on the loss function.
   <2>

The molecular structure optimization system according to <1>, in which the circuit parameter is a rotation angle vector of a quantum gate constituting the parameterized quantum circuit.

<3>

The molecular structure optimization system according to <1> or <2>, in which the coordinate parameter is a vector of coordinates of an atom included in the processing target molecule.

<4>

The molecular structure optimization system according to any one of <1> to <3>, in which the loss function is a Hamiltonian defined by the coordinate parameter of the processing target molecule.

<5>

The molecular structure optimization system according to any one of <1> to <4>, in which the variational optimization procedure is a variational quantum eigenvalue method.

<6>

The molecular structure optimization system according to any one of <1> to <5>, in which the optimization unit determines an optimum value of the coordinate parameter when the processing target molecule has a ground-state molecular structure by minimizing the loss function.

<7>

The molecular structure optimization system according to any one of <1> to <6>, in which the optimization unit determines an optimum value of the coordinate parameter when the processing target molecule has a transition-state molecular structure by maximizing the loss function.

<8>

A molecular structure optimization method includes:
a quantum computing step of using a parameterized quantum circuit defined by a circuit parameter to calculate a loss function from a coordinate parameter of a processing target molecule;
an update step of sequentially updating the coordinate parameter and the circuit parameter based on the loss function; and
an optimization step of repeating a variational optimization procedure including the calculation of the loss function by the quantum computing step and the update of the coordinate parameter and the circuit parameter by the update step until a stop condition is satisfied, and determining an optimum value of the circuit parameter and an optimum value of the coordinate parameter that minimize or maximize the loss function.

The update step includes:
a first update step of estimating a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function; and
a second update step of updating the first provisional value of the first parameter while fixing the second parameter to the second provisional value and changing the first parameter according to the Bayesian optimization algorithm based on the loss function.

The inventions of <2> to <7> described above can be applied to the molecular structure optimization method.

<9>

A parameterized quantum circuit assigned with an optimum value of a circuit parameter determined by a molecular structure optimization method that includes:
a quantum computing step of using a parameterized quantum circuit defined by a circuit parameter to calculate a loss function from a coordinate parameter of a processing target molecule;
an update step of updating the coordinate parameter and the circuit parameter based on the loss function; and
an optimization step of repeating a variational optimization procedure including the calculation of the loss function by the quantum computing step and the update of the coordinate parameter and the circuit parameter by the update step until a stop condition is satisfied, and determining an optimum value of the circuit parameter and an optimum value of the coordinate parameter that minimize or maximize the loss function.

The update step includes
a first update step of estimating a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function; and
a second update step of updating the first provisional value of the first parameter while fixing the second parameter to the second provisional value and changing the first parameter according to the Bayesian optimization algorithm based on the loss function.

The inventions of <2> to <7> described above can be applied to the parameterized quantum circuit.

What is claimed is:

1. A molecular structure optimization system comprising:
a quantum computer that uses a parameterized quantum circuit defined by a circuit parameter to calculate a loss function from a coordinate parameter of a processing target molecule; and
a classical computer including an update unit that updates the coordinate parameter and the circuit parameter based on the loss function, and an optimization unit that repeats a variational optimization procedure including the calculation of the loss function by the quantum computer and the update of the coordinate parameter and the circuit parameter by the update unit until a stop condition is satisfied, and determines an optimum value of the circuit parameter and an optimum value of the coordinate parameter that minimize or maximize the loss function, wherein
the update unit includes:
a first update unit that estimates a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function; and
a second update unit that updates the first provisional value of the first parameter while fixing the second parameter to the second provisional value and changing the first parameter according to the Bayesian optimization algorithm based on the loss function.

2. The molecular structure optimization system according to claim 1, wherein the circuit parameter is a rotation angle vector of a quantum gate constituting the parameterized quantum circuit.

3. The molecular structure optimization system according to claim 1, wherein the coordinate parameter is a vector of coordinates of an atom included in the processing target molecule.

4. The molecular structure optimization system according to claim 1, wherein the loss function is a Hamiltonian defined by the coordinate parameter of the processing target molecule.

5. The molecular structure optimization system according to claim 1, wherein the variational optimization procedure is a variational quantum eigenvalue method.

6. The molecular structure optimization system according to claim 1, wherein the optimization unit determines an optimum value of the coordinate parameter when the processing target molecule has a ground-state molecular structure by minimizing the loss function.

7. The molecular structure optimization system according to claim 1, wherein the optimization unit determines an optimum value of the coordinate parameter when the processing target molecule has a transition-state molecular structure by maximizing the loss function.

8. A molecular structure optimization method comprising:
a quantum computing step of using a parameterized quantum circuit defined by a circuit parameter to calculate a loss function from a coordinate parameter of a processing target molecule;
an update step of sequentially updating the coordinate parameter and the circuit parameter based on the loss function; and
an optimization step of repeating a variational optimization procedure including the calculation of the loss function by the quantum computing step and the update of the coordinate parameter and the circuit parameter by the update step until a stop condition is satisfied, and determining an optimum value of the circuit parameter and an optimum value of the coordinate parameter that minimize or maximize the loss function, wherein
the update step includes:
a first update step of estimating a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function; and
a second update step of updating the first provisional value of the first parameter while fixing the second parameter to the second provisional value and changing the first parameter according to the Bayesian optimization algorithm based on the loss function.

9. A parameterized quantum circuit assigned with an optimum value of a circuit parameter determined by a molecular structure optimization method comprising:
a quantum computing step of using a parameterized quantum circuit defined by a circuit parameter to calculate a loss function from a coordinate parameter of a processing target molecule;
an update step of updating the coordinate parameter and the circuit parameter based on the loss function; and
an optimization step of repeating a variational optimization procedure including the calculation of the loss function by the quantum computing step and the update of the coordinate parameter and the circuit parameter by the update step until a stop condition is satisfied, and determining an optimum value of the circuit parameter and an optimum value of the coordinate parameter that minimize or maximize the loss function, wherein
the update step includes:
a first update step of estimating a second provisional value of a second parameter out of the circuit parameter and the coordinate parameter while fixing a first parameter out of the circuit parameter and the coordinate parameter to a first provisional value and changing the second parameter according to a Bayesian optimization algorithm based on the loss function; and
a second update step of updating the first provisional value of the first parameter while fixing the second parameter to the second provisional value and changing the first parameter according to the Bayesian optimization algorithm based on the loss function.
